(19) United States
(12) Patent Application Publication
(10) Pub. No.: US 2002/0132019 A1
(43) Pub. Date: Sep. 19, 2002

(54) Sterol fractions of Nigella sativa L. seeds

(76) Inventor: Osama Kandil, Herndon, VA (US)

Correspondence Address: Chalin A. Smith, Smith Patent Consulting, LLC, P.O. Box 2726, Alexandria, VA, 22301, US

(21) Appl. No.: 10/029,886

(22) Filed: Dec. 31, 2001

Related U.S. Application Data
(60) Provisional application No. 60/258,555, filed on Dec. 29, 2000.

(51) Int. Cl. A61K 31/56; A61K 35/78

(52) U.S. Cl. 424/764; 514/171

(57) ABSTRACT

The present invention provides novel compositions comprising a Nigella sativa L. sterol fraction. The present invention also provides novel compositions comprising ∃-sitosterol, campesterol, ∃-amyrin, stigmasterol, or any combination thereof. The present invention provides novel methods for treating and preventing fungal infections, bacterial infections, and vaginal diseases and disorders by administering the novel compositions of the invention. The present invention also provides methods for treating or preventing inflammation, pain and/or allergic reactions by administering the novel compositions of the invention.

This application claims the benefit of U.S. Provisional Application, Serial No. 60/258,555, filed Dec. 29, 2000, the contents of which are incorporated by reference herein.

### FIELD OF THE INVENTION

The present invention is generally directed to the field of medicine and pharmacology. More particularly, the present invention is directed to compounds and compositions extracted from Nigella sativa L. seeds, and to methods of using the compounds and compositions.

### BACKGROUND OF THE INVENTION

A variety of herbal and plant extracts are available today for treating many diseases affecting the human body. Some preparations have been known for thousands of years while others are just being discovered to have highly curative effects. Effective plant extracts are highly desired as a natural way to treat diseases. It is believed that natural preparations will not have an adverse effect on the body compared to synthetic preparations.

The Nigella sativa plant is cultivated for its seeds which are used for medicinal purposes. Nigella sativa L seeds come from an herbaceous plant that belongs to the Ranunculaceae family. The plant is a dicotyledon and is cultivated in various parts of the world, especially in Eastern Mediterranean countries and also in India, Bangladesh, Turkey and Pakistan. It is also grown in other places having similar climates, such as East Africa and Middle Europe. The plant is characterized by an erect branched stem and alternating, finely divided, feathery, grayish-green leaves. The bluish-white, star-shaped flowers are terminal and solitary, and there are no petals. The fruit is a globase capsule with small black, rough seeds. Other species of Nigella include Nigella arvensis and Nigella damascena.

The seeds of Nigella sativa are known as Habbatul Baraka (meaning the seed of good fortune) and El Habba El Sawdaa (meaning Black Seed) in the Mediterranean region. The seeds are also known as Kalajira or Kalaoji and Black Cumin in the Indian subcontinent. Nigella sativa L. seeds have been used as a natural remedy for over 4,000 years in various parts of the world, particularly in the Near and Middle East. In the authentic tradition and sayings of the Prophet Muhammad (peace be upon him) which are documented in “Hadeeth” books he is quoted as saying: “In the Black Seed there is healing for every illness except death.”

There is a need in the art for new pharmaceutical compounds and compositions that are derived from natural sources, like plants. The present invention is directed to this, as well as other, important ends.

### SUMMARY OF THE INVENTION

The present invention provides novel compositions comprising a Nigella sativa L. sterol fraction. The present invention also provides novel methods for treating and preventing fungal infections, bacterial infections, and vaginal diseases and disorders by administering an effective amount of the novel compositions of the invention. These and other aspects of the present invention are described in detail herein.

### BRIEF DESCRIPTION OF THE FIGURE

FIG. 1 shows the process for preparing the Nigella sativa L. sterol fraction of the present invention from Nigella sativa L. seeds.

FIG. 2 shows the percent concentration of components present in Nigella sativa L. seeds after the various treatment and extractions described in FIG. 1.

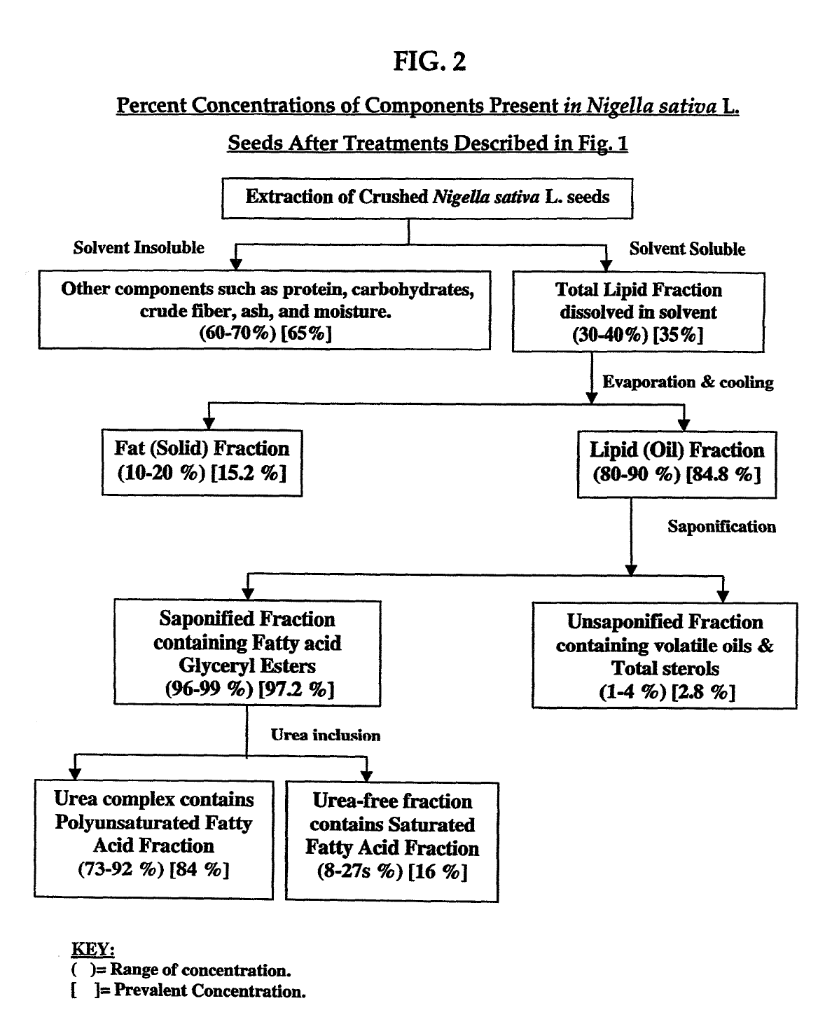

### DETAILED DESCRIPTION OF THE INVENTION

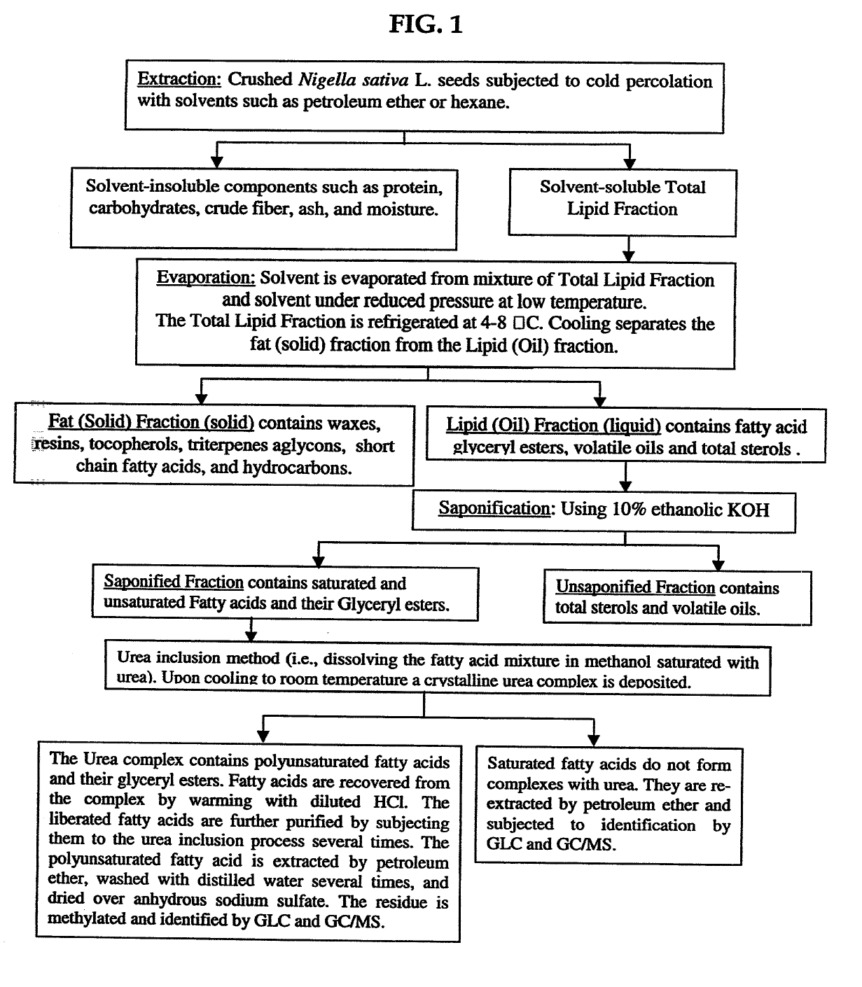

In one embodiment, the present invention describes novel compositions comprising a Nigella sativa L. sterol fraction. A “Nigella sativa L. sterol fraction” refers to the sterol fraction extracted from the seeds of Nigella sativa L. The “Nigella sativa L. sterol fraction” can be extracted from the seeds of Nigella sativa L. following the methods described in Example 1 and FIG. 1. Preferably, the “Nigella sativa L. sterol fraction” comprises ∃-sitosterol, campesterol, stigmasterol and ∃-amyrin. ∃-sitosterol is the main component of the Nigella sativa L. sterol fraction.

In other embodiments, the present invention provides novel compositions comprising ∃-sitosterol, campesterol, ∃-amyrin, stigmasterol or any combination thereof. “Compositions comprising ∃-sitosterol, campesterol, ∃-amyrin, stigmasterol or any combination thereof” refers to the following embodiments. In one embodiment, the composition may comprise ∃-sitosterol. In another embodiment, the composition may comprise campesterol. In another embodiment, the composition may comprise ∃-amyrin.

In another embodiment, the composition may comprise stigmasterol. In yet another embodiment, the composition may comprise ∃-sitosterol and B-amyrin. In yet another embodiment, the composition may comprise ∃-sitosterol and stigmasterol. In yet another embodiment, the composition may comprise ∀-amyrin and stigmasterol. In yet another embodiment, the composition may comprise ∃-sitosterol and campesterol. In yet another embodiment, the composition may comprise stigmasterol and campesterol. In yet another embodiment, the composition may comprise ∃-amyrin and campesterol. In still other embodiments, the composition may comprise ∃-sitosterol, campesterol, and ∃-amyrin. In still other embodiments, the composition may comprise ∃-sitosterol, campesterol, and stigmasterol. In still other embodiments, the composition may comprise ∃-sitosterol, ∃-amyrin, and stigmasterol. In still other embodiments, the composition may comprise campesterol, ∃-amyrin, and stigmasterol. In preferred embodiments, the composition comprises ∃-sitosterol, campesterol, B-amyrin, and stigmasterol.

In one embodiment, the present invention provides compositions comprising the Nigella sativa L. sterol fraction described herein in an amount of about 0.1 to about 25% by weight, based on 100% by weight of the total composition. Preferably, the Nigella sativa L. sterol fraction described herein is present in an amount of about 1 to about 20% by weight; more preferably in an amount of about 2 to about 15% by weight; still more preferably in an amount of about 3 to about 10% by weight, still more preferably in an amount of about 4 to about 6% by weight; most preferably in an amount of about 5% by weight.

In a preferred embodiment, the present invention provides compositions comprising the Nigella sativa L. sterol fraction described herein in an amount of about 0.5 to about 25% by weight, based on 100% by weight of the total composition. Preferably, the Nigella sativa L. sterol fraction described herein is present in an amount of about 1 to about 20% by weight; more preferably in an amount of about 5 to about 15% by weight; still more preferably in an amount of about 7 to about 13% by weight, still more preferably in an amount of about 9 to about 11% by weight; most preferably in an amount of about 10% by weight.

The present invention provides novel methods for modulating fungal growth by administering an effective amount of a composition comprising the Nigella sativa L. sterol fraction of the present invention or by administering an effective amount of a composition comprising ∃-sitosterol, campesterol, ∃-amyrin, stigmasterol or any combination thereof. The growth of any fungi known in the art can be modulated with the compositions of the present invention. Exemplary fungi whose growth can be modulated include, but are not limited to, those from the genus Candida (e.g., Candida albicans), the genus Microsporum (e.g., Microsporum canis, Microsporum gypseum), the genus Aspergillus (e.g., Aspergillus flavus, Aspergillus niger), the genus Penicillium (e.g., Penicillium spp), the genus Tinea (e.g., Tinea circinata, Tineapedis, Tinea cruris, Tinea versicolor, Tinea axillaris, Tinea capitis), the genus Monilia, the genus Cladosporium, the genus Phialophora, and the genus Paracoccidioides (e.g., Paracoccidioides brasiliensis).

“Modulating fungal growth” refers to killing or eliminating the fungi, reducing the amount of fungi (e.g., compared to the amount of fungi present before the composition of the present invention was applied), or slowing the rate of growth of the fungi (e.g., compared to the rate of growth of the fungi absent the presence of the composition of the present invention).

The present invention also provides novel methods for treating and preventing fungal infections in a patient in need thereof by administering an effective amount of a composition comprising the Nigella sativa L. sterol fraction of the present invention or by administering an effective amount of a composition comprising ∃-sitosterol, campesterol, ∃-amyrin, stigmasterol or any combination thereof. Any fungal infection known in the art can be treated or prevented with the compositions of the present invention. Preferably, the fungal infection is a fungal skin infection. Exemplary fungal infections that can be prevented or treated with the compositions of the present invention include, but are not limited to, those from the genus Candida (e.g., Candida albicans, Candida tropicalis, Candida glabrata, Candida parapsilosis), the genus Microsporum (e.g., Microsporum canis, Microsporum gypseum), the genus Aspergillus (e.g., Aspergillus flavus, Aspergillus niger), the genus Penicillium (e.g., Penicillium spp), the genus Tinea (e.g., Tinea circinata, Tineapedis, Tinea cruris, Tinea versicolor, Tinea axillaris, Tinea capitis), the genus Monilia, the genus Cladosporium, the genus Phialophora, and the genus Paracoccidioides (e.g., Paracoccidioides brasiliensis). One skilled in the art will appreciate that Dermatophytoses (e.g., ring worm/tinea) affect, for example, skin, hair and nails; Aspergillus affect, for example, ear, eyes, lungs and skin, and Candida affect, for example, the respiratory tract, the gastrointestinal tract, the urogenital tract (e.g., the vulva and vagina), and skin.

“Treating and preventing fungal infections” includes eliminating or curing the fungal infection, reducing the size or severity of the fungal infection (e.g., compared to the size or severity of the fungal infection before the compositions of the present invention were administered), and/or reducing the rate of growth of the fungal infection (e.g., compared to the rate of growth of the fungal infection in the absence of the compositions of the present invention). The term “patient” refers to animals, preferably mammals, more preferably humans, and includes infants, children and adults.

In a preferred embodiment, the present invention is directed to methods of treating fungal infections or modulating fungal growth where the fungus is Candida albicans or Monilia spp. by administering the Nigella sativa L. sterol fraction described herein. The Nigella sativa L. sterol fraction of the present invention is particularly effective against fungi that are resistant to conventional antifungal drugs such as, for example, vaginal moniliasis.

The present invention provides novel methods for modulating bacterial growth by administering an effective amount of a composition comprising the Nigella sativa L. sterol fraction of the present invention or by administering an effective amount of a composition comprising ∃-sitosterol, campesterol, ∃-amyrin, stigmasterol or any combination thereof. The growth of any bacteria known in the art can be modulated with the compositions of the present invention. Exemplary bacteria whose growth can be modulated include, but are not limited to, those from the genus Staphylococcus (e.g., Staphyloccus aureus, staphyloccus pyogenes), Corynebacterium (e.g., Corynebacterium pyogenes), Streptococcus (e.g., Streptococcus pyogenes), Salmonella (e.g., Salmonella typhi murium), Escherichia (e.g., Escherichia coli), Pseudomonas (e.g., Pseudomonas aeroginosa), and Klebsiella (e.g., Klebsiella spp.).

“Modulating bacterial growth” refers to killing or eliminating the bacteria, reducing the amount of bacteria (e.g., compared to the amount of bacteria present before the composition of the present invention was applied), or slowing the rate of growth of the bacteria (e.g., compared to the rate of growth of the bacteria absent the presence of the composition of the present invention).

The present invention also provides novel methods for treating and preventing bacterial infections in a patient in need thereof by administering an effective amount of a composition comprising the Nigella sativa L. sterol fraction of the present invention or by administering an effective amount of a composition comprising ∃-sitosterol, campesterol, ∃-amyrin, stigmasterol or any combination thereof. Any bacterial infection known in the art can be treated or prevented with the compositions of the present invention. Exemplary bacterial infections that can be prevented or treated with the compositions of the present invention include, but are not limited to, those from the genus Staphylococcus (e.g., Staphyloccus aureus, staphyloccus pyogenes), Corynebacterium (e.g., Corynebacterium pyogenes), Streptococcus (e.g., Streptococcus pyogenes), Salmonella (e.g., Salmonella typhi murium), Escherichia (e.g., Escherichia coli), Pseudomonas (e.g., Pseudomonas aeroginosa), and Klebsiella (e.g., Klebsiella spp.).

“Treating and preventing bacterial infections” includes eliminating or curing the bacterial infection, reducing the severity of the bacterial infection (e.g., compared to the severity of the bacterial infection before the compositions of the present invention were administered), and/or reducing the rate of growth of the bacterial infection (e.g., compared to the rate of growth of the fungal infection in the absence of the compositions of the present invention). As used throughout the disclosure, the term “patient” refers to animals, preferably mammals, more preferably humans, and includes infants, children and adults, and males and females.

The present invention provides methods for treating and preventing vaginal diseases and disorders by administering an effective amount of a composition comprising the Nigella sativa L. sterol fraction of the present invention or by administering an effective amount of a composition comprising ∃-sitosterol, campesterol, ∃-amyrin, stigmasterol or any combination thereof. The composition is preferably administered as a topical composition (e.g., lotion, cream, ointment or gel) a vaginal suppository, or a urethral suppository. The composition is preferably administered to the vagina or vulva.

“Vaginal diseases and disorders” includes, but is not limited to, vaginal moniliasis, vaginitis, vaginal discharge, vaginal itching, vaginal inflammation, candidiasis (vaginal and vulvovaginal), vulvovaginitis, bacterial vaginitis, trichomonas vaginitis, cervicitis, vaginal irritation, pruritis, vaginal burning, atrophic vaginitis, sexual transmitted diseases, and the like.

“Vaginal itching” may be caused by allergies or other inflammatory conditions of the vagina including, for example, chemical irritation and irritation from tight clothing, and inflammation caused by direct local trauma, douching, harsh soaps, perfumed toilet paper or pads, or contraceptive agents. Sterol and/or sterolin combinations have been experimentally demonstrated to reduce the secretion of the pro-inflammatory cytokines controlled by the Th₂ helper cells and to increase the number of Th₁ cells which regulate the secretion of antibodies from B cells, providing relief and/or a cure from vaginal itching and/or vaginal inflammation

“Bacterial vaginitis” is can be caused by any bacteria, and is generally caused by Gardnerella vaginalis, Corynebacterium vaginale or Hemophilus vaginalis. Bacterial vaginitis is common in cases of sexual abuse and is characterized by tissue inflammation, bacterial infections, burning, itching, a grayish discharge, and an odor.

“Sexually transmitted disease,” as used herein, generally refers to herpes, chlamydia, and gonorrhea.

Vaginal discharge, pruritis, vaginal burning, and candidiasis (vaginal or vulvovaginal) are generally caused by sexual activity, medications (such as contraceptive drugs and corticosteroids) or feminine hygiene products.

Trichomonas vaginitis (also known as Trichomonas vaginalis) is a flagellated protozoan that generally lives in the vagina and urethra. It can be carried asymptomatically in both men and women, particularly in the postmenopausal period, and is transmitted through sexual intercourse.

“Vulvovaginitis” can be any known in the art and includes, for example, fungal vulvovaginitis and chemical vulvovaginitis. Fungal vulvovaginitis can be caused by candidiasis or monilia (including Candida albicans, Candida tropicalis, Candida glabrata & Candida parapsilosis). Chemical vulvovaginitis is caused by the direct contact of the vulvovaginal area with chemicals (such as soaps, perfumes, toilet paper or pads, powders, contraceptive agents, feminine hygiene products, or strong vinegar douches). Chemical vulvovaginitis can be present in children due to harsh soaps or bubble baths or trauma to the genital area.

“Cervicitis” also known as endocervicitis is an inflammation of the endocervical mucosa. “Atrophic vaginitis” is an inflammation of the vaginal epithelium due to lack of estrogen and is characterized by burning, dryness, soreness, and vaginal irritation. It is most common in postmenopausal women and nursing mothers. The effectiveness of the composition of the present invention in treating and/or preventing cervicitis and atrophic vaginitis may be due to its structural similarities with estrogen.

The present invention also provides a novel method for treating and preventing vaginal moniliasis in immunocompromised patients by administering to patients in need thereof a therapeutically effective amount of a composition comprising the Nigella sativa L. sterols fraction of the present invention or by administering an effective amount of a composition comprising ∃-sitosterol, ∃-amyrin, stigmasterol or any combination thereof. The immunocompromised patients may have AIDS, HIV infection, or a transplant. Sterols/sterolins play an important immunomodulatory role to control inflammation. Bouic et al, International Journal of Immunopharmacology, 18(12):643-700 (1996); Bouic, Newsletter of the Arthritis Trust of America (Summer 1998). The composition is preferably administered as a topical composition or a vaginal suppository.

The present invention also provides methods for increasing permeability and integrity of cell membranes and normalizing the functions of cell membranes (e.g., maintaining the physiological role of cell membranes) by administering an effective amount of a composition comprising the Nigella sativa L. sterol fraction of the present invention or by administering an effective amount of a composition comprising ∃-sitosterol, campesterol, ∃-amyrin, stigmasterol or any combination thereof. The present invention is also directed to a preparation a pharmaceutical formula to ensure fluidity and stability of skin cell membrane as it is a structural component of cell membranes.

The present invention provides methods for treating or preventing inflammation, pain, and/or allergic reactions in a patient in need thereof by administering an effective amount of a composition comprising the Nigella sativa L. sterol fraction of the present invention or by administering an effective amount of a composition comprising ∃-sitosterol, campesterol,∃-amyrin, stigmasterol or any combination thereof.

The dosage regimen for treating the diseases described herein is selected in accordance with a variety of factors, including the age, weight, sex, and medical condition of the patient, the severity of the disease, the route of administration, pharmacological considerations such as the activity, efficacy, pharmacokinetic and toxicology profiles of the particular compound or composition used, whether a drug delivery system is used and whether the compound or composition is administered as part of a drug combination.

The compounds and compositions of the present invention can be administered orally, topically, parenterally, by inhalation (nasal or oral), vaginally, urethrally, or rectally in dosage unit formulations containing conventional nontoxic pharmaceutically acceptable carriers, adjuvants, and vehicles, as desired. One skilled in the art will appreciate that the compounds described herein may be present in the form of various conventional pharmaceutically acceptable salts. The term parenteral as used herein includes subcutaneous, intravenous, intramuscular, intrasternal injection, or infusion techniques. Preferably, the compounds or compositions of the present invention are topically administered, more preferably they are topically administered to the vagina or vulva.

Injectable preparations, for example, sterile injectable aqueous or oleaginous suspensions may be formulated according to the known art using suitable dispersing or wetting agents, suspending agents (e.g., methylcellulose, Polysorbate 80, hydroxyethylcellulose, acacia, powdered tragacanth, sodium carboxymethylcellulose, polyoxytehylene sorbitan monolaurate and the like), pH modifiers, buffers, solubilizing agents (e.g., polyoxyethylene hydrogenated castor oil, Polysorbate 80, nicotinamide, polyoxyethylene sorbitan monolaurate, Macrogol, an ethyl ester of castor oil fatty acid, and the like) and preservatives. The sterile injectable preparation may also be a sterile injectable solution or suspension in a nontoxic parenterally acceptable diluent or solvent, for example, as a solution in 1,3-butanediol. Among the acceptable vehicles and solvents that may be used are water, Ringer's solution, and isotonic sodium chloride solution. In addition, sterile, total lipids are conventionally used as a solvent or suspending medium. For this purpose any bland total lipid may be used including synthetic mono- or diglycerides, in addition, fatty acids such as oleic acid find use in the preparation of injectables. The preparations can be lyophilized by methods known in the art.

Solid dosage forms for oral administration may include capsules, soft gel capsules, tablets, sublingual tablets, powders, granules and gels. In such solid dosage forms, the active compound(s) may be admixed with one or more inert diluents such as lactose or starch. As is normal practice, such dosage forms may also comprise other substances including lubricating agents such as magnesium stearate. In the case of capsules, tablets, and pills, the dosage forms may also comprise buffering agents. The tablets can be prepared with enteric or film coatings.

Liquid dosage forms for oral administration can include pharmaceutically acceptable emulsions, solutions, suspensions, and syrups containing inert diluents commonly used in the art, such as water. Such compositions can also comprise adjuvants, such as wetting agents, emulsifying and suspending agents, and sweetening, flavoring, and perfuming agents. When pharmaceutical preparations of the present invention are prepared for treating infants or children, they are preferably prepared in a liquid dosage form.

For administration by inhalation (oral or nasal), the compositions of the invention can be delivered from an insufflator, a nebulizer or a pressured pack or other convenient mode of delivering an aerosol spray. Pressurized packs can include a suitable propellant. Alternatively, for administration by inhalation, the compositions can be administered in the form of a dry powder composition or in the form of a liquid spray.

Suppositories for vaginal, urethral, or rectal administration can be prepared by mixing the active compounds with suitable nonirritating excipients such as cocoa butter and polyethylene glycols that are solid at room temperature and liquid at body temperature.

For topical administration, the compounds or compositions of the invention can be formulated as ointments, creams, gels, or lotions, or as the active ingredient of a transdermal patch. Topical administration can also be accomplished with a liquid spray, an aerosol, or via iontophoresis, or through the use of liposomes, microbubbles and/or microcapsules. Ointments and creams may be formulated, for example, with an aqueous or oily base with the addition of suitable thickening (e.g., PEG 4000, PEG 6000, wax, hard paraffin) and/or gelling agents (e.g., hydroxypropyl cellulose). Lotions may be formulated with an aqueous or oily base and can also generally contain one or more emulsifying agents (e.g., wool wax alcohol, fatty acid glycol esters), stabilizing agents (e.g., polyoxyethylene sorbitan monolaurate, carboxy methyl cellulose), dispersing agents (e.g., sodium oleate, propylene glycol), suspending agents (e.g., methyl cellulose, chitosan, accacia, carboxymethyl cellulose, tragacanth, pectin), thickening agents, and/or coloring agents (e.g., dyes, lackes). Topical administration includes administration to the vulva and vagina.

Generally, a lotion is a suspension of finely divided active ingredient in a small amount of water. Lotions provide fast release of the active ingredient due to the soluble nature of the active ingredient and the water content. Lotions generally have short duration of action. Generally, an ointment is a semi-solid preparation that is more viscous and provides for slow release of the active ingredients due to longer duration of contact with the skin. Generally, a cream is a semi-solid preparation that contains a humactant and a higher percentage of water than an ointment. Creams are less viscous than ointments and provide for release of the active ingredients over a moderate period of time.

While the compounds of the invention can be administered as the sole active pharmaceutical agent in the methods described herein, they can also be used in combination with one or more compounds which are known to be therapeutically effective against the specific disease that one is targeting for treatment.

The present invention is described in terms of a Nigella sativa L. sterol fraction. The methods for obtaining a Nigella sativa L. sterol fraction can also be followed to obtain a Nigella arvensis sterol fraction, a Nigella damascena sterol fraction, or a sterol fraction from any other species of Nigella. Moreover, a Nigella arvensis sterol fraction and/or a Nigella damascena sterol fraction can be used for modulating fungal growth, treating and preventing fungal infections, modulating bacterial growth, treating and preventing bacterial infections, treating and preventing vaginal diseases and disorders, for increasing permeability and integrity of cell membranes and normalizing the functions of cell membranes, and for treating or preventing inflammation, pain or allergic reactions, as described herein.

### EXAMPLES

The following examples are for purposes of illustration only, and are not intended to limit the scope of the appended claims.

### Example 1

A process for preparing the Nigella sativa L. sterol fraction of the present invention is outlined in FIG. 1, and is described herein.

Nigella sativa L. seeds were crushed and extracted in a percolator until exhaustion with petroleum ether (40-60° C.) or hexane. The petroleum ether or hexane extract was evaporated under reduced pressure at 40° C. and the residue was kept in a refrigerator. The saturated fatty acids fraction and waxes solidified upon cooling and were separated by filtration or decantation.

100 grams of the remaining oil(after removal of the separated solid), was subjected to saponification with 10% ethanolic KOH and benzene, then the solvent was distilled off to dryness. The residue was mixed with water and extracted with diethyl ether several times. The combined ether extracts was washed with distilled water till free from alkalinity, filtered, and then dried in a desiccator over anhydrous sodium sulfate. The residue obtained after distillation of ether was dried in a deseccator over anhydrous CaCl₂. The unsaponifiable matter (2.7 grams) is a yellowish semisolid and showed crystalline deposits.

Chromatographic investigation using TLC, CC and preparative TLC, indicated the presence of four main components: ∃-sitosterol, campesterol, ∃-amyrin, and stigmasterol, in addition to other components which were isolated in small amounts.

Thin layer chromatography (TLC) was performed. The ether solution of the unsaponified materials was spotted alongside authentic chloroformic solutions of ∃-sitosterol, alfa-amyrin and ∃-amyrin (0.1%) on silica gel G plates using the following solvent systems: (system 1) benzene:ethyl acetate (3:1) v/v; (system 2) benzene:ethyl acetate (86:14) v/v. The developed plates were then dried, sprayed with 50% sulfuric acid in alcohol and heated at 110° C. for 10 minutes. The results are shown below.

TABLE 1

| | | | Color of spot after | |
|---|---|---|---|---|
| | | | spray with 50% | |
| | RF Values | | sulfuric acid | |
| Spot No. | System 1 | System 2 | in alcohol | Authentication |
| 1 | 0.13 | 0.17 | blue | unidentified |
| 2 | 0.50 | 0.56 | violet | ∃-sitosterol, |
| | | | | campesterol |
| | | | | and |
| | | | | stigmasterol |
| 3 | 0.64 | 0.71 | yellowish-brown | ∃-amyrin |
| 4 | 0.85 | 0.89 | brown | unidentified |

The results of the TLC investigation of the Nigella sativa L. sterol fraction revealed the presence of four main spots on the two solvent systems. Spot Nos. 2 and 3 were chromatographically identical to those of authentic ∃-sitosterol and ∃-amyrin. Spot Nos. 1 and 4 could not be identified with TLC. The components in spot nos. 1 and 4 were present in small quantities and may contain steryl sterols and/or steryl glucosides.

Column chromatography(CC) was also performed on the Nigella sativa L. sterol fraction. Fractions of about 3 grams of the Nigella sativa L. sterol fraction were dissolved in 10 ml chloroform(CHCl₃) and mixed with 5 grams neutral alumina to form a homogenous mass. The mass was applied on the top of a glass column containing slurry of 60 grams of neutral alumina in petroleum ether. Elution was carried out with solvents of increasing polarity including petroleum ether(boiling range of 40-60° C.), containing increasing amounts of benzene. Twenty ml fractions were collected and monitored by TLC using the solvent system benzene:ethyl acetate (86:14 v/v). Spots were located using 50% methanolic sulfuric acid followed by heating at 100° C. for 10 minutes. The results are presented in table 2 below.

TABLE 2

| | | | Lieber- | | | |
|---|---|---|---|---|---|---|
| | | | man's | | | |
| | | Fraction | Burchard | No. of | | |
| No | Eluent | Number | Test | Spots | R_{F} | Color |
| 1 | pet. ether | 1-20 | − | — | — | — |
| | (100) | | | | | |
| 2 | pet ether: | 21-35 | − | — | — | — |
| | benzene | | | | | |
| | (95:5) | | | | | |
| 3 | pet ether: | 36-45 | − | — | — | — |
| | benzene | | | | | |
| | (90:10) | | | | | |
| 4 | pet ether: | 46-53 | + | 3 | 0.34 | brown |
| | benzene | | | | 0.88 | yellow-brown |
| | (85:15) | | | | 0.72 | brown |
| 5 | pet ether: | 54-70 | + | 2 | 0.88 | yellow-brown |
| | benzene | | | | 0.72 | brown |
| | (80:20) | | | | | |
| 6 | pet ether: | 71-82 | + | 2 | 0.72 | brown |
| | benzene | | | | 0.56 | violet |
| | (70:30) | | | | | |
| 7 | pet ether: | 83-95 | + | 1 | 0.56 | violet |
| | benzene | | | | | |
| | (60:40) | | | | | |
| 8 | Pet ether: | 96-120 | + | 1 | 0.56 | Violet |
| | benzene | | | | | |
| | (50:50) | | | | | |
| 9 | Pet ether: | 121-140 | − | — | — | — |
| | benzene | | | | | |
| | (40:60) | | | | | |

The residue left after evaporation of the solvent from fractions 54-70 and +ve Lieberman's Burchard test was crystallized from absolute methanol and revealed white needle crystals, which were dried over anhydrous CaCl₂ in a deseccator to afford a pure crystal with a melting point of 186-187° C. Using TLC and a solvent system of benzene:ethyl acetate (86:14 v/v) and an IR-Spectrum (KBr disc) and mixed melting points revealed the presence of ∃-amyrin.

Fractions 83-120 were also crystallized from absolute methanol to yield white needle crystals and +ve Lieberman's Burchard test. Using the same solvent system as above, the IR-Spectrum, and a melting point of 137-139° C. indicated the presence of ∃-sitosterol.

Further identification was performed through acetylation of the final products, purification, crystallization, examination of the acetylated product and examination by TLC using silica gel G impregnated with 10% AgNO₃. The loaded plates were loped with the solvent system petroleum ether: chloroform: acetic acid (75:25:0.5). Three spots were observed which were identical to those of authentic ∃-sitosterol, stigmasterol and campesterol.

### Example 2

### In vitro Studies To Evaluate the Anti-monilial Effects Of the Nigella sativa L. Sterol Fraction

The antifungal activity of the Nigella sativa L. sterol fraction was detected in vitro using the zone of inhibition. Sabouraud's glucose agar medium at 45° C. was poured into sterilized petri-dishes, solidified by cooling and then one central bore was made by means of sterilized borer No. 5.

Different concentrations of the Nigella sativa L. sterol fraction was poured into the bores and each concentration was tested in 2 petri dishes. A loop full of spore suspension from each fungus was inoculated by stabbing with a wire needle above the central bore. Petri dishes were then incubated at 30° C. for 7 days for molds, 21 days for dermatophytes and 2 days for yeast, and then the diameter of colonies in each plate was measured.

The data in the table below shows that the Nigella sativa L. sterol fraction of the present invention was able to inhibit the growth of Candida albicans in a dose dependent manner.

TABLE 3

| | Concentration | Zone of inhibition |
|---|---|---|
| Microorganism | (Mg/ml) | (mm) |
| Candida albicans | 1 | 13.0 ± 0.16 |
| | 5 | 17.2 ± 0.33 |
| | 10 | 21.7 ± 0.57 |
| | 25 | 30.1 ± 0.88 |
| | 50 | 39.5 ± 1.87 |

### Example 3

### In vivo Studies to Evaluate the Anti-Allergic Effects Of the Nigella sativa L. Sterol Fraction

The prophylactic anti-allergic effects of the Nigella sativa L. sterol fraction were evaluated in guinea pigs (300-400 grams). Animals were divided into 5 groups (10 animals each) and treated i.p. As follows:

First: Animals treated by saline.

Second: Animals treated by diphenhydramine (239 mg/kg/day).

Third: Animals treated by the Nigella sativa L. sterol fraction (50 mg/kg).

Fourth:

Animals treated by the Nigella sativa L. sterol fraction (100 mg/kg).

Fifth: Animals treated by the Nigella sativa L. sterol fraction (150mg/kg).

Animals were exposed 30 minutes after their i.p. Injection of diphenhydramine and the Nigella sativa L. sterol fraction (50,100 and 150 mg/kg) to histamine aerosol (1% for 3 minutes to induce bronchospasm or tachypnea) (Armitage et al, Br. J. Pharmacol., 16:59-76 (1961)). The percent protection against bronchospasm and loss of rightening after 30, 60 and 90 minutes was calculated (Dawson and Sweatman, Br. J. Pharmacol, 71:387-398 (1980)).

The table below shows the prophylactic effects of the Nigella sativa L. sterol fraction on histamine-induced bronchospasm in guinea pigs (data are the mean observations from 10 animals). The Nigella sativa L. sterol fraction at 100 mg/kg and 150 mg/kg protected guinea pigs from histamine-induced bronchospasm.

TABLE 4

| | Dose | % protection | % protection | % protection |
|---|---|---|---|---|
| Group | (mg/kg) | 30 minutes | 60 minutes | 90 minutes |
| Control | | 0 | 0 | 0 |
| Diphenhydramine | 239 | 100 | 100 | 90 |
| Nigella sativa L. | 50 | 0 | 0 | 0 |
| sterol fraction | | | | |
| Nigella sativa L. | 100 | 70 | 70 | 60 |
| sterol fraction | | | | |
| Nigella sativa L. | 150 | 90 | 90 | 80 |
| sterol fraction | | | | |

### Example 4 Vaginal Suppositories

A pharmaceutical formulation was prepared containing 10% of the Nigella sativa L. L. sterol fraction of the present invention as a vaginal suppository containing 0.4 grams Nigella sativa L. sterol fraction in a base of 3.6 grams of polyethylene glycol (PEG). The PEG was melted to 50° C., then 0.4 grams of the Nigella sativa L. sterol fraction was added thereto. The mixture was poured into a vaginal suppository mold, allowed to cool, cleaned and dispensed.

A second pharmaceutical formulation was prepared containing 5% of the Nigella sativa L. sterol fraction of the present invention as a vaginal suppository containing 0.2 grams Nigella sativa L. sterol fraction in each suppository in a base of 4.8 grams of polyethylene glycol (PEG). The PEG was melted to 50° C., then 0.2 grams of the Nigella sativa L. sterol fraction was added thereto. The mixture was poured into a vaginal suppository mold, allowed to cool, cleaned and dispensed.

### Example 5

### Phase I Clinical Trials

Ten healthy female volunteers were selected for this study. Volunteers were screened according to their medical history (including concomitant medication history), physical examination and laboratory investigations (e.g. chest X-ray, ECG, liver and kidney function tests, urine for glucosuria), a thorough skin examination, and local vaginal examination including a vaginal smear.

The healthy female volunteers with ages ranging from 25 to 35 years were free from vaginal diseases and had different complexions (fair and dark). Any volunteers having vaginal lesions or positive vaginal smears, diabetes and neurological disorders, or with emotional or psychological instability, were excluded from the study. Prior to the study each volunteer signed a consent form.

Subjects were asked to apply the composition in Example 4 containing 10% Nigella sativa L. sterol fraction (twice daily for one week) topically (the vaginal suppository is considered “topical” as it is not a systemic application), and were hospitalized for 48 hours where all vital signs were monitored and laboratory investigations (including liver & kidney function tests, hematological studies, urine and stool analysis in addition to an ECG and a chest X-ray) were done immediately before and by the end of the period of administration of the composition in Example 4. On the 8th day, they were readmitted for clinical and laboratory evaluation. Subjects were allowed to attend the outpatient clinic of the hospital once weekly for check ups.

The results of the study revealed no local irritation or sensitization in the volunteers. Physical examination and laboratory investigations carried out at the end of the week revealed no statistically different changes. The composition in Example 4 was well tolerated in all the volunteers enrolled in this trial without any observable local or systemic side effects.

### Example 6

### Phase II Clinical Trials

The aim of this work was to study the effectiveness of the composition in Example 4 in the treatment of vaginal moniliasis through an open trial. Vaginal moniliasis patients were selected from the outpatient clinics of some hospitals in Cairo, Egypt. Patients were chosen according to the clinical diagnosis. 47 patients completed the trials.

All patients were screened according to their medical history (including concomitant medication history), and a physical examination and laboratory investigations (including urine for glucosuria and a vaginal smear). Patients having vaginal moniliasis, and patients who were married or previously married in the childbearing period with ages ranging from 25 to 35 years were included in the study. Patients were excluded from the study if they were diabetic as indicated by renal glucosuria, had emotional or psychological instability, had hepatic or renal diseases, or were taking immunosuppressive agents including corticosteroids.

Patients willing to participate in the clinical trial had to complete a physical examination and laboratory investigations. Patients enrolled in the study were given the vaginal suppositories described in Example 4 twice daily (morning and evening) for one week. A vaginal smear was performed on all patients at the end of the trial to assure the absence of candida. Patients were followed up once weekly. Patients' urine was tested for glucosuria. Prior to the trial, a signed consent was obtained from the patients.

Results of the study, presented in the table below for the composition containing 10% Nigella sativa L. sterol fraction, showed that 30 patients exhibited complete cure (64%) from vaginal moniliasis, 14 patients were improved (30%) from the vaginal moniliasis, and 3 patients did not improve (6%). A complete cure was a clinical and mycological cure (scraping and culture). Improvement was marked relief of symptoms and signs of the disease (vaginal smear may still be positive). No response was minimal clinical and mycological improvement occurred.

TABLE 5

| | Complete Cure | | Improvement | | Minimal Response | |
|---|---|---|---|---|---|---|
| # of | # of | % | # of | % | # of | % |
| patients | patients | response | patients | response | patients | response |
| 47 | 30 | 64 | 14 | 30 | 3 | 6 |

Both experimental and clinical trials (phases I & II) confirmed that the vaginal suppositories in Example 4 were a potent and safe formula that successfully treated vaginal moniliasis. In vitro mycological studies proved antimonilial activity against Candida albicans. Additionally, the Nigella sativa L. sterol fraction had anti-allergic and anti-inflammatory effects as proved by protection from histamine-induced bronchospasm in guinea pigs.

The phase I clinical trial demonstrated that local application of the suppositories in Example 4 twice daily for one week is quite safe without the incidence of any side reactions or toxicity in all subjects enrolled in the trial.

The phase II clinical studies demonstrated that the vaginal suppositories in Example 4 were effective in the treatment of vaginal moniliasis. The 10% Nigella sativa L. L. sterol fraction vaginal suppository in Example 4 achieved a 94 % success (64% total cure and 30% improvement) and a 6% minimal response in the treatment of vaginal moniliasis. The suppositories in Example 4 are considered equal to conventional drugs utilized in the treatment of vaginal moniliasis.

The vaginal suppositories in Example 4 possessed a safe antimonilial formula. Phase I and II clinical trials indicated that the suppositories in Example 4 were safe and efficacious in the treatment of vaginal moniliasis.

### Example 7

This example explains an in vivo study to evaluate the prophylactic (protective) anti-asthmatic effect of the Nigella sativa L. sterol fraction. The prophylactic anti-asthmatic effect was evaluated in vivo by measurement of the pre-convulsive time in guinea pigs exposed to histamine aerosol (0.25%).

Guinea pigs were divided into three groups with 8 animals per group. The first group received saline injections to serve as negative control. The second group received ketotifen, which is the standard treatment to serve as positive control. The third group received 200 mg/kg each of the Nigella sativa L. sterol fraction of the present invention.

Animals in all groups were treated for 21 days after which time they were exposed to histamine (0.25 %) aerosol and the preconvulsive time was recorded. The results are shown in the table below.

TABLE 6

| | Preconvulsive time | % of Prophylaxis | |
|---|---|---|---|
| Treatment | mean ± SD (seconds) | (Protection) | Dosage |
| saline | 71.6 ± 14.6 | 11.9 | |
| negative control | | | |
| ketotifen | 497.1* ± 183.2 | 82.85 | 50 :g/kg |
| positive standard | | | |
| Nigella sativa L. | 338* ± 146 | 56.7 | 200 mg/kg |
| Sterol Fraction | | | |

*Statistically significant from the control group (P < 0.05)

### Example 8

This experiment was an in vivo study to evaluate the tachypnea (protective) effect of the Nigella sativa L. sterol fraction of the present invention.

The second protocol involved sensitized animals. Animals were sensitized by injecting ova albumin in two doses 100 mg. each (S.C. and I.P., simultaneously). Animals were resensitized 15 days later. Guinea pigs were divided into three groups with 8 animals per group. The first group received a saline injection to serve as negative control. The second group received ketotifen which is the standard treatment to serve as positive control. The third group received 200 mg/kg each of the Nigella sativa L. sterol fraction of the present invention.

Animals in all groups were treated for 21 days following which they were exposed to ova albumin inhalation and time to onset of tachypnea was recorded as shown in the table below.

TABLE 7

| | Time to attain | | |
|---|---|---|---|
| | Tachypnea mean ± SD | | |
| Treatment | (seconds) | % of Prophylaxis | Dosage |
| saline - | 81.4 ± 33.3 | 13.6 | |
| negative control | | | |
| ketotifen | 266.3* ± 128 | 44.4 | 50 :g/kg |
| positive standard | | | |
| Nigella sativa L. | 213.8* ± 99.4 | 35.6 | 200 mg/kg |
| Sterol Fraction | | | |

*Statistically significant from the control group (P < 0.05)

Each of the patents, applications, and publications cited herein is incorporated by reference herein in their entirety.

It will be apparent to one skilled in the art that various modifications can be made to the invention without departing from the spirit or scope of the appended claims.

What is claimed is:

1. A composition comprising a Nigella sativa L. sterol fraction.

2. The composition of claim 1, wherein the Nigella sativa L. sterol fraction is present in the composition in an amount of about 0.5 to about 25% by weight based on 100% by weight of the total composition.

3. The composition of claim 2, wherein the Nigella sativa L. sterol fraction is present in an amount of about 5% by weight.

4. The composition of claim 2, wherein the Nigella sativa L. sterol fraction is present in an amount of about 10% by weight.

5. The composition of claim 1, wherein the Nigella sativa L. sterol fraction comprises ∃-sitosterol, campesterol, ∃-amyrin and stigmasterol.

6. A vaginal suppository comprising a Nigella sativa L. sterol fraction.

7. A topical composition comprising a Nigella sativa L. sterol fraction.

8. The topical composition of claim 7, wherein the topical composition is a lotion, a cream, an ointment or a gel.

9. A composition comprising ∃-sitosterol, campesterol, ∃-amyrin and stigmasterol.

10. A method of treating or preventing a fungal infection in a patient in need thereof comprising topically administering an effective amount of the composition in claim 1 in a pharmaceutically acceptable carrier.

11. A method of treating or preventing a bacterial infection in a patient in need thereof comprising topically administering an effective amount of the composition in claim 1 in a pharmaceutically acceptable carrier.

12. A method of treating or preventing a vaginal disease or disorder in a patient in need thereof comprising administering an effective amount of the composition in claim 1 in a pharmaceutically acceptable carrier.

13. The method of claim 12, wherein the vaginal disease or disorder is vaginal moniliasis, vaginitis, vaginal discharge, vaginal itching, vaginal inflammation, candidiasis, vulvovaginitis, bacterial vaginitis, trichmonas vaginitis, cervicitis, vaginal irritation, pruritis, vaginal burning, atrophic vaginitis or a sexual transmitted disease.

14. The method of claim 12, wherein the composition is administered topically.

15. The method of claim 12, wherein the composition is administered as a suppository.

16. A method for treating or preventing vaginal moniliasis in an immuno compromised patient comprising administering an effective amount of the composition of claim 1 in a pharmaceutically acceptable carrier.

16. A method for treating or preventing inflammation, pain or an allergic reaction in a patient in need thereof comprising administering an effective amount of the composition of claim 1 in a pharmaceutically acceptable carrier.

17. A method for increasing permeability and integrity of a cell membrane or normalizing the function of a cell membrane by administering an effective amount of the composition of claim 1.

18. A method for modulating bacterial or fungal growth comprising applying a composition comprising an effective amount of a Nigella sativa L. sterol fraction.
